SEQUENCE LISTING (64 sequences)

SEQ ID NO: 1 - protein, 419 aa, Artificial Sequence
  Synthetic
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 422 aa, Artificial Sequence
  Synthetic
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 3 - protein, 413 aa, Artificial Sequence
  Synthetic
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 4 - protein, 1273 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1255 aa, Artificial Sequence
  Synthetic
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 6 - protein, 1353 aa, Artificial Sequence
  Synthetic
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 7 - protein, 7 aa, Artificial Sequence
  Synthetic
DVNGYVA

SEQ ID NO: 8 - protein, 8 aa, Artificial Sequence
  Synthetic
YFTSSLYS

SEQ ID NO: 9 - protein, 7 aa, Artificial Sequence
  Synthetic
HFSFPFT

SEQ ID NO: 10 - protein, 108 aa, Artificial Sequence
  Synthetic
DIQMTQSPSSLSASVGDRVTITCRASQDVNGYVAWYQQKPGKAPKLLIYFTSSLYSGVPSRFSGSGSGTDFTLTISSLQPEDFATYYCQQHFSFPFTFGQGTKVEIKR

SEQ ID NO: 11 - protein, 9 aa, Artificial Sequence
  Synthetic
FTINTWGIH

SEQ ID NO: 12 - protein, 10 aa, Artificial Sequence
  Synthetic
GILPYGGHSD

SEQ ID NO: 13 - protein, 4 aa, Artificial Sequence
  Synthetic
ATLM

SEQ ID NO: 14 - protein, 111 aa, Artificial Sequence
  Synthetic
EVQLVESGGGLVQPGGSLRLSCAASGFTINTWGIHWVRQAPGKGLEWVAGILPYGGHSDYADSVKGRFTISADTSKNTAYLQMNSLRAEDTAVYYCATLMRGQGTLVTVSS

SEQ ID NO: 15 - protein, 7 aa, Artificial Sequence
  Synthetic
DVSSAVA

SEQ ID NO: 16 - protein, 8 aa, Artificial Sequence
  Synthetic
YTTNWLLS

SEQ ID NO: 17 - protein, 7 aa, Artificial Sequence
  Synthetic
FYDYPIT

SEQ ID NO: 18 - protein, 108 aa, Artificial Sequence
  Synthetic
DIQMTQSPSSLSASVGDRVTITCRASQDVSSAVAWYQQKPGKAPKLLIYTTNWLLSGVPSRFSGSGSGTDFTLTISSLQPEDFATYYCQQFYDYPITFGQGTKVEIKR

SEQ ID NO: 19 - protein, 9 aa, Artificial Sequence
  Synthetic
FSIDAGAIH

SEQ ID NO: 20 - protein, 10 aa, Artificial Sequence
  Synthetic
GITPYGGKTY

SEQ ID NO: 21 - protein, 13 aa, Artificial Sequence
  Synthetic
ARWAWGLRSGMDY

SEQ ID NO: 22 - protein, 120 aa, Artificial Sequence
  Synthetic
EVQLVESGGGLVQPGGSLRLSCAASGFSIDAGAIHWVRQAPGKGLEWVAGITPYGGKTYYADSVKGRFTISADTSKNTAYLQMNSLRAEDTAVYYCARWAWGLRSGMDYWGQGTLVTVSS

SEQ ID NO: 23 - protein, 7 aa, Artificial Sequence
  Synthetic
DVNNWVA

SEQ ID NO: 24 - protein, 8 aa, Artificial Sequence
  Synthetic
YFTSYLYS

SEQ ID NO: 25 - protein, 7 aa, Artificial Sequence
  Synthetic
HSTYPFT

SEQ ID NO: 26 - protein, 108 aa, Artificial Sequence
  Synthetic
DIQMTQSPSSLSASVGDRVTITCRASQDVNNWVAWYQQKPGKAPKLLIYFTSYLYSGVPSRFSGSGSGTDFTLTISSLQPEDFATYYCQQHSTYPFTFGQGTKVEIKR

SEQ ID NO: 27 - protein, 9 aa, Artificial Sequence
  Synthetic
FTITDYGIH

SEQ ID NO: 28 - protein, 10 aa, Artificial Sequence
  Synthetic
YIGPSDGDTH

SEQ ID NO: 29 - protein, 13 aa, Artificial Sequence
  Synthetic
ARTGGYNSWYMDY

SEQ ID NO: 30 - protein, 120 aa, Artificial Sequence
  Synthetic
EVQLVESGGGLVQPGGSLRLSCAASGFTITDYGIHWVRQAPGKGLEWVAYIGPSDGDTHYADSVKGRFTISADTSKNTAYLQMNSLRAEDTAVYYCARTGGYNSWYMDYWGQGTLVTVSS

SEQ ID NO: 31 - protein, 7 aa, Artificial Sequence
  Synthetic
DVWYYVA

SEQ ID NO: 32 - protein, 8 aa, Artificial Sequence
  Synthetic
SRTNWLDS

SEQ ID NO: 33 - protein, 7 aa, Artificial Sequence
  Synthetic
YSDYPLT

SEQ ID NO: 34 - protein, 108 aa, Artificial Sequence
  Synthetic
DIQMTQSPSSLSASVGDRVSITCRASQDVWYYVAWYQQKPGKAPKLLISRTNWLDSGVPSRFSGSGSGTDFTLTISSLQPEDFATYYCQQYSDYPLTFGQGTKVEIKR

SEQ ID NO: 35 - protein, 9 aa, Artificial Sequence
  Synthetic
FTITNWWIH

SEQ ID NO: 36 - protein, 10 aa, Artificial Sequence
  Synthetic
AISPFNGKTH

SEQ ID NO: 37 - protein, 9 aa, Artificial Sequence
  Synthetic
ARLLSIYDY

SEQ ID NO: 38 - protein, 116 aa, Artificial Sequence
  Synthetic
EVQLVESGGGLVQPGGSLRLSCAASGFTITNWWIHWVRQAPGKGLEWVAAISPFNGKTHYADSVKGRFTISADTSKNTAYLQMNSLRAEDTAVYYCARLLSIYDYWGQGTLVTVSS

SEQ ID NO: 39 - protein, 7 aa, Artificial Sequence
  Synthetic
DVRNFVA

SEQ ID NO: 40 - protein, 8 aa, Artificial Sequence
  Synthetic
YYTRYLYS

SEQ ID NO: 41 - protein, 7 aa, Artificial Sequence
  Synthetic
FSTFPFT

SEQ ID NO: 42 - protein, 108 aa, Artificial Sequence
  Synthetic
DIQMTQSPSSLSASVGDRVTITCRASQDVRNFVAWYQQKPGKAPKLLIYYTRYLYSGVPSRFSGSGSGTDFTLTISSLQPEDFATYYCQQFSTFPFTFGQGTKVEIKR

SEQ ID NO: 43 - protein, 9 aa, Artificial Sequence
  Synthetic
FTIGGRFIH

SEQ ID NO: 44 - protein, 10 aa, Artificial Sequence
  Synthetic
GIYPYGGHSS

SEQ ID NO: 45 - protein, 9 aa, Artificial Sequence
  Synthetic
ASGVTYMDY

SEQ ID NO: 46 - protein, 115 aa, Artificial Sequence
  Synthetic
EVQLVESGGGLVQPGGSLRLSCAASGFTIGGRFIHWVRQAPGKGLEWVAGIYPYGGHSSYADSVKGRFTISADTSKNTAYLQMNSLRAEDTAVYYCASGVTYMDYWGQGTLVTVS

SEQ ID NO: 47 - protein, 7 aa, Artificial Sequence
  Synthetic
DVSNGVA

SEQ ID NO: 48 - protein, 8 aa, Artificial Sequence
  Synthetic
SGANWLES

SEQ ID NO: 49 - protein, 7 aa, Artificial Sequence
  Synthetic
FYNYPLT

SEQ ID NO: 50 - protein, 108 aa, Artificial Sequence
  Synthetic
DIQMTQSPSSLSASVGDRVTITCRASQDVSNGVAWYQQKPGKAPKLLISGANWLESGVPSRFSGSGSGTDFTLTISSLQPEDFATYYCQQFYNYPLTFGQGTKVEIKR

SEQ ID NO: 51 - protein, 9 aa, Artificial Sequence
  Synthetic
FTINDWGIH

SEQ ID NO: 52 - protein, 10 aa, Artificial Sequence
  Synthetic
GIWPYGGSTF

SEQ ID NO: 53 - protein, 13 aa, Artificial Sequence
  Synthetic
ARWMGGLRSAMDY

SEQ ID NO: 54 - protein, 120 aa, Artificial Sequence
  Synthetic
EVQLVESGGGLVQPGGSLRLSCAASGFTINDWGIHWVRQAPGKGLEWVAGIWPYGGSTFYADSVKGRFTISADTSKNTAYLQMNSLRAEDTAVYYCARWMGGLRSAMDYWGQGTLVTVSS

SEQ ID NO: 55 - protein, 7 aa, Artificial Sequence
  Synthetic
NVHNGVA

SEQ ID NO: 56 - protein, 8 aa, Artificial Sequence
  Synthetic
SGTNWLPS

SEQ ID NO: 57 - protein, 7 aa, Artificial Sequence
  Synthetic
YFGYPLT

SEQ ID NO: 58 - protein, 108 aa, Artificial Sequence
  Synthetic
DIQMTQSPSSLSASVGDRVTITCRASQNVHNGVAWYQQKPGKAPKLLISGTNWLPSGVPSRFSGSGSGTDFTLTISSLQPEDFATYYCQQYFGYPLTFGQGTKVEIKR

SEQ ID NO: 59 - protein, 9 aa, Artificial Sequence
  Synthetic
FTINDGGIH

SEQ ID NO: 60 - protein, 10 aa, Artificial Sequence
  Synthetic
AINPFNGNTY

SEQ ID NO: 61 - protein, 13 aa, Artificial Sequence
  Synthetic
AKWTGVSRSSMDY

SEQ ID NO: 62 - protein, 120 aa, Artificial Sequence
  Synthetic
EVQLVESGGGLVQPGGSLRLSCAASGFTINDGGIHWVRQAPGKGLEWVAAINPFNGNTYYADSVKGRFTISADTSKNTAYLQMNSLRAEDTAVYYCAKWTGVSRSSMDYWGQGTLVTVSS

SEQ ID NO: 63 - protein, 106 aa, Artificial Sequence
  Synthetic
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 118 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELL

(19) United States
(12) Patent Application Publication
(10) Pub. No.: US 2021/0341478 A1
(43) Pub. Date: Nov. 4, 2021

(54) RECOMBINANT ANTIBODIES, KITS COMPRISING THE SAME, AND USES THEREOF

(71) Applicant: AnTaimmu BioMed Co., Ltd., hsinchu City (TW)

(72) Inventors: San-Tai SHEN, Miaoli County (TW); Shih-Hu HO, Hsinchu City (TW); Ing-Chien CHEN, Hsinchu City (TW)

(73) Assignee: AnTaimmu BioMed Co., Ltd., Hsinchu City (TW)

(21) Appl. No.: 17/241,355

(22) Filed: Apr. 27, 2021

Related U.S. Application Data
(60) Provisional application No. 63/016,987, filed on Apr. 29, 2020.

(51) Int. Cl. G01N 33/569; C07K 16/10; G01N 33/563

(52) U.S. Cl. CPC G01N 33/56983; C07K 16/10; G01N 2333/165; C07K 2317/565; G01N 33/563

(57) ABSTRACT

Disclosed herein is a recombinant antibody against the nucleocapsid protein of severe acute respiratory syndrome coronavirus 2 (SARS-CoV-2). A kit containing the recombinant antibody for use in coronavirus detection is encompassed in the present disclosure. Also disclosed herein are in vitro methods of detecting coronavirus infection in a subject with the aid of the recombinant antibody.

### CROSS-REFERENCE OF RELATED APPLICATION

This application claims priority to U.S. Application No. 63/016,987, filed on Apr. 29, 2020. The content of which application is incorporated herein by reference in its entirety.

### BACKGROUND OF THE INVENTION

### 1. Field of the Invention

The present disclosure in general relates to the field of coronavirus detection. More particularly, the present disclosure relates to recombinant antibodies against coronaviruses.

### 2. Description of Related Art

Coronaviruses are a group of enveloped positive-sense RNA viruses that cause illness ranging from the common cold to severe respiratory tract infections, including Middle East respiratory syndrome coronavirus (MERS-CoV), severe acute respiratory syndrome coronavirus (SARS-CoV), and severe acute respiratory syndrome coronavirus 2 (SARS-CoV-2; or 2019 coronavirus disease (COVID-19)). Coronaviruses is featured by the club-shape spike projecting from the surface of the virion, and the virus particles are constituted by four main structural proteins, including the spike (S), membrane (M), envelope (E), and nucleocapsid (N) proteins, all of which are encoded within the 3′ end of the viral genome.

Since the outbreak of COVID-19 in the end of 2019, COVID-19 has been rapidly spread globally. Up to date at the end of 2020, there are more than 84,000,000 COVID-19 cases had been confirmed, and more than 1,800,000 deaths had been reported around the world. An important measure for the control of spread of the pandemic relies on a rapid and robust detection of the coronavirus infection in a suspected case in the first place. However, most of the detection measurements at current use (e.g., virus culture, nucleic acid test, antigen test, or antibody test) are time-consuming, costly, with accuracy and precision far below satisfactory.

In view of the foregoing, there exists in the related art a need for an improved method for rapid detection of coronavirus infection.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a recombinant antibody specific to a coronavirus, which comprises a light chain variable (VL) region and a heavy chain variable (VH) region, wherein the VL region comprises a first complementarity determining region (CDR-L1), a second CDR (CDR-L2), and a third CDR (CDR-L3), and the VH region comprises a first complementarity determining region (CDR-H1), a second CDR (CDR-H2), and a third CDR (CDR-H3), wherein

the CDR-L1, the CDR-L2, the CDR-L3, the CDR-H1, the CDR-H2, and the CDR-H3 respectively comprise the amino acid sequences of SEQ ID NOs: 7, 8, 9, 11, 12, and 13;

the CDR-L1, the CDR-L2, the CDR-L3, the CDR-H1, the CDR-H2, and the CDR-H3 respectively comprise the amino acid sequences of SEQ ID NOs: 15, 16, 17, 19, 20, and 21;

the CDR-L1, the CDR-L2, the CDR-L3, the CDR-H1, the CDR-H2, and the CDR-H3 respectively comprise the amino acid sequences of SEQ ID NOs: 23, 24, 25, 27, 28, and 29;

the CDR-L1, the CDR-L2, the CDR-L3, the CDR-H1, the CDR-H2, and the CDR-H3 respectively comprise the amino acid sequences of SEQ ID NOs: 31, 32, 33, 35, 36, and 37;

the CDR-L1, the CDR-L2, the CDR-L3, the CDR-H1, the CDR-H2, and the CDR-H3 respectively comprise the amino acid sequences of SEQ ID NOs: 39, 40, 41, 43, 44, and 45;

the CDR-L1, the CDR-L2, the CDR-L3, the CDR-H1, the CDR-H2, and the CDR-H3 respectively comprise the amino acid sequences of SEQ ID NOs: 47, 48, 49, 51, 52, and 53; or

the CDR-L1, the CDR-L2, the CDR-L3, the CDR-H1, the CDR-H2, and the CDR-H3 respectively comprise the amino acid sequences of SEQ ID NOs: 55, 56, 57, 59, 60, and 61.

According to one specific embodiment of the present disclosure, the present recombinant antibody has the CDR-L1, the CDR-L2, the CDR-L3, the CDR-H1, the CDR-H2, and the CDR-H3 respectively comprising the amino acid sequences of SEQ ID NOs: 23, 24, 25, 27, 28, and 29.

According to some embodiments of the present disclosure, the present recombinant antibody has

the VL and the VH regions respectively comprising the amino acid sequences of SEQ ID NOs: 10 and 14;

the VL and the VH regions respectively comprising the amino acid sequences of SEQ ID NOs: 18 and 22;

the VL and the VH regions respectively comprising the amino acid sequences of SEQ ID NOs: 26 and 30;

the VL and the VH regions respectively comprising the amino acid sequences of SEQ ID NOs: 34 and 38;

the VL and the VH regions respectively comprising the amino acid sequences of SEQ ID NOs: 42 and 46;

the VL and the VH regions respectively comprising the amino acid sequences of SEQ ID NOs: 50 and 54; or

the VL and the VH regions respectively comprising the amino acid sequences of SEQ ID NOs: 58 and 62.

According to one specific embodiment of the present disclosure, the present recombinant antibody has the VL and the VH regions respectively comprising the amino acid sequences of SEQ ID NOs: 26 and 30.

Optionally, the present recombinant antibody may be conjugated with a reporter molecule or a nanoparticle.

Exemplary reporter molecule is acridine orange, acridine yellow, alkaline phosphatase (AP), auramine, benzoxadiazole, bilirubin, biotin, blue fluorescent protein (BFP), 6′-carboxyfluorescein (FAM), cascade blue, cresyl violet, crystal violet, cyan fluorescent protein (CFP), cyanine, DNA probe, eosin, fluorescein, fluorescein isothiocyanate, glutathione-S-transferase (GST), green fluorescence protein (GFP), horseradish peroxidase (HRP), indocarbocyanine, malachite green, merocyanine, Nile blue, Nile red, nitrobenzoxadiazole, orotidine 5′-phosphate decarboxylase, oxacarbocyanine, peridinin chlorophyll, phycoerythrin, phthalocyanine, porphine, proflavine, pyridyloxazole, red fluorescent protein (RFP), rhodamine, thiacarbocyanine, thioredoxin (TRX), or yellow fluorescent protein (YFP).

The nanoparticle suitable for use in the present recombinant antibody is aluminium oxide particle, boron particle, calcium particle, carbon nanotube, cerium oxide particle, clay particle, copper particle, diamond particle, gold particle, graphene particle, hydroxy acid particle, hydroxyapatite particle, iron particle, kojic acid particle, liposome, manganese particle, molybdenum particle, palladium particle, platinum particle, phosphorus particle, potassium particle, silicon dioxide particle, silver particle, sodium silicate particle, titanium dioxide particle, ytterbium trifluoride particle, zinc particle, zinc oxide particle, or zirconium dioxide particle.

According to one specific embodiment of the present disclosure, the present recombinant antibody is specific to SARS-CoV-2.

Another aspect of the present disclosure pertains to a kit for detecting the infection of coronavirus.

The present kit comprises a first and a second recombinant antibodies. According to some embodiments of the present disclosure, the first recombinant antibody has the CDR-L1, the CDR-L2, the CDR-L3, the CDR-H1, the CDR-H2, and the CDR-H3 respectively comprising the amino acid sequences of SEQ ID NOs: 23, 24, 25, 27, 28, and 29. In these embodiments, the second recombinant antibody has

the CDR-L1, the CDR-L2, the CDR-L3, the CDR-H1, the CDR-H2, and the CDR-H3 respectively comprising the amino acid sequences of SEQ ID NOs: 7, 8, 9, 11, 12, and 13;

the CDR-L1, the CDR-L2, the CDR-L3, the CDR-H1, the CDR-H2, and the CDR-H3 respectively comprising the amino acid sequences of SEQ ID NOs: 15, 16, 17, 19, 20, and 21;

the CDR-L1, the CDR-L2, the CDR-L3, the CDR-H1, the CDR-H2, and the CDR-H3 respectively comprising the amino acid sequences of SEQ ID NOs: 31, 32, 33, 35, 36, and 37;

the CDR-L1, the CDR-L2, the CDR-L3, the CDR-H1, the CDR-H2, and the CDR-H3 respectively comprising the amino acid sequences of SEQ ID NOs: 39, 40, 41, 43, 44, and 45;

the CDR-L1, the CDR-L2, the CDR-L3, the CDR-H1, the CDR-H2, and the CDR-H3 respectively comprising the amino acid sequences of SEQ ID NOs: 47, 48, 49, 51, 52, and 53; or

the CDR-L1, the CDR-L2, the CDR-L3, the CDR-H1, the CDR-H2, and the CDR-H3 respectively comprising the amino acid sequences of SEQ ID NOs: 55, 56, 57, 59, 60, and 61.

According to some embodiments of the present disclosure, in the present kit, the first recombinant antibody has the VL and the VH regions respectively comprising the amino acid sequences of SEQ ID NO: 26 and 30; and the second recombinant antibody has

the VL and the VH regions respectively comprising the amino acid sequences of SEQ ID NO: 10 and 14;

the VL and the VH regions respectively comprising the amino acid sequences of SEQ ID NO: 18 and 22;

the VL and the VH regions respectively comprising the amino acid sequences of SEQ ID NO: 34 and 38;

the VL and the VH regions respectively comprising the amino acid sequences of SEQ ID NO: 42 and 46;

the VL and the VH regions respectively comprising the amino acid sequences of SEQ ID NO: 50 and 54; or

the VL and the VH regions respectively comprising the amino acid sequences of SEQ ID NO: 58 and 62.

Optionally, the present kit further comprises a solid phase support, wherein one of the first and the second recombinant antibodies is linked to the solid phase support, and the other recombinant antibody is conjugated with a reporter molecule or a nanoparticle. Examples of the solid phase support include, but are not limited to, a plate, a bead, a tube, a filter, a chip, a film, and the like.

Also encompassed in the present disclosure is a method of determining whether a subject is infected by a coronavirus; the method is performed on a biological sample isolated from the subject, and comprises the step of, detecting the presence or absence of a nucleocapsid protein of the coronavirus in the biological sample by use of the present recombinant antibody, wherein the presence of the nucleocapsid protein indicates that the subject is infected by the coronavirus.

According to some embodiments of the present disclosure, the biological sample may be blood, plasma, serum, saliva, sputum, urine, tissue (e.g., biopsy, materials from a nasal swab, or materials from a throat swab), or tissue lysate.

According to some examples of the present disclosure, the coronavirus is SARS-CoV-2.

Preferably, the subject is a human.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIG. 1 is the results of ELISA that depicts the binding of specified single chain fragment variable (scFv), including NP-1 to NP-7, toward the nucleocapsid protein of SARS-CoV-2 expressed by Escherichia coli (E. coli). IgG isotype was used as a negative control.

FIG. 2 is the results of ELISA that depicts the binding of specified immunoglobulin G (IgG), including NP-1 to NP-7, toward the nucleocapsid protein of SARS-CoV-2 expressed by E. coli.

FIG. 3 is the results of ELISA that depicts the binding of specified immunoglobulin G (IgG), including NP-1 to NP-7, toward the nucleocapsid protein of SARS-CoV-2 expressed in HEK-293 cells (293-NP, as indicated). HEK-293 cells without the nucleocapsid protein expression (293-WT, as indicated) were used as a binding control, and IgG isotype was used as a negative control.

FIG. 4 depicts the standard curves of NP-3 (panel A) and NP-4 (panel B) IgG for binding to different amounts of the nucleocapsid protein of SARS-CoV-2 as determined by ELISA.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of molecular biology, microbiology, recombinant DNA, and immunology, which are within the skill of the art. Such techniques are explained fully in the literature.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “recombinant antibody,” as used herein, refers to antibodies that are prepared, expressed, created, or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created, or isolated by any other means that involves splicing of human immunoglobulin gene sequences to other DNA sequences. Such recombinant antibodies include humanized, CDR grafted, chimeric, in vitro generated (e.g., by phage display) antibodies, and may optionally include constant regions derived from human germline immunoglobulin sequences. Also, “recombinant antibody” may direct to a portion of an intact antibody, including, without limitation, Fv, Fab, Fab′, F(ab′)2, diabodies, scFv, and single domain antibodies (e.g., variable heavy domain (VHH)).

The “variable region” or “variable domain” of an antibody refers to the amino-terminal domains of heavy or light chain of the antibody. These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The term “complementarity determining region” (CDR) used herein refers to the hypervariable region of an antibody molecule that forms a surface complementary to the 3-dimensional surface of a bound antigen. Proceeding from N-terminus to C-terminus, each of the antibody heavy and light chains comprises three CDRs (CDR 1, CDR 2, and CDR3). An antigen-binding site, therefore, includes a total of six CDRs that comprise three CDRs from the variable region of a heavy chain and three CDRs from the variable region of a light chain.

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100$$

where X is the number of amino acid residues scored as identical matches by the sequence alignment program BLAST in that program's alignment of A and B, and where Y is the total number of amino acid residues in A or B, whichever is shorter.

The terms “conjugated” or “conjugate” are used herein to refer to two or more entities that are linked by direct or indirect covalent or non-covalent interaction. In some embodiments, the present recombinant antibody is conjugated with a reporter molecule (e.g., a fluorescent molecule). In other embodiments, the present recombinant antibody is conjugated with a nanoparticle (e.g., a gold particle).

The terms “treatment” and “treating” as used herein may refer to a curative or palliative measure. In particular, the term “treating” as used herein refers to the application or administration of an antiviral agent to a subject, who has a coronavirus infectious disease, a symptom associated with a coronavirus infectious disease, a disease or disorder secondary to a coronavirus infectious disease, with the purpose to partially or completely alleviate, ameliorate, relieve, delay onset of, inhibit progression of, reduce severity of, and/or reduce incidence of one or more symptoms or features of a coronavirus infectious disease.

The term “an effective amount” as used herein refers to an amount effective, at dosages, and for periods of time necessary, to achieve the desired therapeutically desired result with respect to the treatment of coronavirus infection.

The term “administered,” “administering” or “administration” are used interchangeably herein to refer to a mode of delivery, including, without limitation, intravenously, intramuscularly, intraperitoneally, intraarterially, intracranially, or subcutaneously administering an antiviral agent to a subject having coronavirus infection. In some embodiments, the antiviral agent is mixed with a suitable excipient (e.g., buffer solution) before use, such as intravenous injection.

The term “subject” or “patient” refers to an animal including the human species that is treatable with the method of the present disclosure. The term “subject” or “patient” intended to refer to both the male and female gender unless one gender is specifically indicated. Accordingly, the term “subject” or “patient” comprises any mammal which may benefit from treatment of coronavirus infection. Examples of a “subject” or “patient” include, but are not limited to, human, rat, mouse, guinea pig, monkey, pig, goat, cow, horse, dog, cat, bird and fowl. In one exemplary embodiment, the patient is a mouse. In another exemplary embodiment, the patient is a human.

### II. Description of the Invention

Provided herein are seven recombinant antibodies specific to the nucleocapsid protein of the coronavirus, and uses of the recombinant antibodies in detecting coronavirus. Due to the conservativeness of the nucleocapsid protein among many species of the coronavirus family, some of the recombinant antibodies may recognize different species of the coronavirus, including MERS-CoV, SARS-CoV, and SARS-CoV-2. Some of the recombinant antibodies, on the other hand, specifically bind to SARS-CoV-2, and do not cross-react with other coronaviruses.

1. The Recombinant Antibody

The first aspect of the present disclosure is directed to seven recombinant antibodies respectively designated as NP-1 to NP-7 scFvs or IgGs. According to embodiments of the present disclosure, each of NP-1 to NP-7 scFvs or IgGs comprises a VL region and a VH region, wherein the VL region comprises three CDRs (i.e., CDR-L1, CDR-L2 and CDR-L3), and the VH region comprises three CDRs (i.e., CDR-H1, CDR-H2 and CDR-H3).

According to some embodiments of the present disclosure, the CDR-L1, CDR-L2, CDR-L3 of the recombinant antibody NP-1 scFv or IgG respectively comprise the amino acid sequences of SEQ ID NOs: 7, 8 and 9; and the CDR-H1, CDR-H2, and CDR-H3 of the recombinant antibody NP-1 scFv or IgG respectively comprise the amino acid sequences of SEQ ID NOs: 11, 12 and 13. Preferably, the VL region of the recombinant antibody NP-1 scFv or IgG comprises the amino acid sequence at least 80% (i.e., 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 10, and the VH region of the recombinant antibody NP-1 scFv or IgG comprises the amino acid sequence at least 80% (i.e., 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 14. As could be appreciated, the framework sequence of the VL and VH regions may vary (e.g., being substituted by conserved or non-conserved amino acid residues) without affecting the binding affinity and/or specificity of the present antibody. Preferably, the sequences of the framework is conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue). According to the preferred embodiment, the VL and VH regions of the recombinant antibody NP-1 scFv or IgG respectively comprise the amino acid sequences at least 85% identical to SEQ ID NOs: 10 and 14. More preferably, the VL and VH regions of the recombinant antibody NP-1 scFv or IgG respectively comprise the amino acid sequences at least 90% identical to SEQ ID NOs: 10 and 14. Even more preferably, the VL and VH regions of the recombinant antibody NP-1 scFv or IgG respectively comprise the amino acid sequences at least 95% identical to SEQ ID NOs: 10 and 14. In one working example of the present disclosure, the VL region of the recombinant antibody NP-1 scFv or IgG has the amino acid sequence of SEQ ID NO: 10, and the VH region of the recombinant antibody NP-1 scFv or IgG has the amino acid sequence of SEQ ID NO: 14.

In certain embodiments, the CDR-L1, CDR-L2, CDR-L3 of the recombinant antibody NP-2 scFv or IgG respectively comprise the amino acid sequences of SEQ ID NOs: 15, 16 and 17; and the CDR-H1, CDR-H2, and CDR-H3 of the recombinant antibody NP-2 scFv or IgG respectively comprise the amino acid sequences of SEQ ID NOs: 19, 20 and 21. Preferably, the VL region of the recombinant antibody NP-2 scFv or IgG comprises the amino acid sequence at least 80% identical to SEQ ID NO: 18, and the VH region of the recombinant antibody NP-2 scFv or IgG comprises the amino acid sequence at least 80% identical to SEQ ID NO: 22. According to the preferred embodiment, the VL and VH regions of the recombinant antibody NP-2 scFv or IgG respectively comprise the amino acid sequences at least 85% identical to SEQ ID NOs: 18 and 22. More preferably, the VL and VH regions of the recombinant antibody NP-2 scFv or IgG respectively comprise the amino acid sequences at least 90% identical to SEQ ID NOs: 18 and 22. Even more preferably, the VL and VH regions of the recombinant antibody NP-2 scFv or IgG respectively comprise the amino acid sequences at least 95% identical to SEQ ID NOs: 18 and 22. In one working example of the present disclosure, the VL region of the recombinant antibody NP-2 scFv or IgG has the amino acid sequence of SEQ ID NO: 18, and the VH region of the recombinant antibody NP-2 scFv or IgG has the amino acid sequence of SEQ ID NO: 22.

Further, the CDR-L1, CDR-L2, CDR-L3 of the recombinant antibody NP-3 scFv or IgG respectively comprise the amino acid sequences of SEQ ID NOs: 23, 24 and 25; and the CDR-H1, CDR-H2, and CDR-H3 of the recombinant antibody NP-3 scFv or IgG respectively comprise the amino acid sequences of SEQ ID NOs: 27, 28 and 29. Preferably, the VL region of the recombinant antibody NP-3 scFv or IgG comprises the amino acid sequence at least 80% identical to SEQ ID NO: 26, and the VH region of the recombinant antibody NP-3 scFv or IgG comprises the amino acid sequence at least 80% identical to SEQ ID NO: 30. According to the preferred embodiment, the VL and VH regions of the recombinant antibody NP-3 scFv or IgG respectively comprise the amino acid sequences at least 85% identical to SEQ ID NOs: 26 and 30. More preferably, the VL and VH regions of the recombinant antibody NP-3 scFv or IgG respectively comprise the amino acid sequences at least 90% identical to SEQ ID NOs: 26 and 30. Even more preferably, the VL and VH regions of the recombinant antibody NP-3 scFv or IgG respectively comprise the amino acid sequences at least 95% identical to SEQ ID NOs: 26 and 30. In one working example of the present disclosure, the VL region of the recombinant antibody NP-3 scFv or IgG has the amino acid sequence of SEQ ID NO: 26, and the VH region of the recombinant antibody NP-3 scFv or IgG has the amino acid sequence of SEQ ID NO: 30.

According to alternative embodiments of the present disclosure, the CDR-L1, CDR-L2, CDR-L3 of the recombinant antibody NP-4 scFv or IgG respectively comprise the amino acid sequences of SEQ ID NOs: 31, 32 and 33; and the CDR-H1, CDR-H2, and CDR-H3 of the recombinant antibody NP-4 scFv or IgG respectively comprise the amino acid sequences of SEQ ID NOs: 35, 36 and 37. Preferably, the VL region of the recombinant antibody NP-4 scFv or IgG comprises the amino acid sequence at least 80% identical to SEQ ID NO: 34, and the VH region of the recombinant antibody NP-4 scFv or IgG comprises the amino acid sequence at least 80% identical to SEQ ID NO: 38. According to the preferred embodiment, the VL and VH regions of the recombinant antibody NP-4 scFv or IgG respectively comprise the amino acid sequences at least 85% identical to SEQ ID NOs: 34 and 38. More preferably, the VL and VH regions of the recombinant antibody NP-4 scFv or IgG respectively comprise the amino acid sequences at least 90% identical to SEQ ID NOs: 34 and 38. Even more preferably, the VL and VH regions of the recombinant antibody NP-4 scFv or IgG respectively comprise the amino acid sequences at least 95% identical to SEQ ID NOs: 34 and 38. In one working example of the present disclosure, the VL region of the recombinant antibody NP-4 scFv or IgG has the amino acid sequence of SEQ ID NO: 34, and the VH region of the recombinant antibody NP-4 scFv or IgG has the amino acid sequence of SEQ ID NO: 38.

In addition, the CDR-L1, CDR-L2, CDR-L3 of the recombinant antibody NP-5 scFv or IgG respectively comprise the amino acid sequences of SEQ ID NOs: 39, 40 and 41; and the CDR-H1, CDR-H2, and CDR-H3 of the recombinant antibody NP-5 scFv or IgG respectively comprise the amino acid sequences of SEQ ID NOs: 43, 44 and 45. Preferably, the VL region of the recombinant antibody NP-5 scFv or IgG comprises the amino acid sequence at least 80% identical to SEQ ID NO: 42, and the VH region of the recombinant antibody NP-5 scFv or IgG comprises the amino acid sequence at least 80% identical to SEQ ID NO: 46. According to the preferred embodiment, the VL and VH regions of the recombinant antibody NP-5 scFv or IgG respectively comprise the amino acid sequences at least 85% identical to SEQ ID NOs: 42 and 46. More preferably, the VL and VH regions of the recombinant antibody NP-5 scFv or IgG respectively comprise the amino acid sequences at least 90% identical to SEQ ID NOs: 42 and 46. Even more preferably, the VL and VH regions of the recombinant antibody NP-5 scFv or IgG respectively comprise the amino acid sequences at least 95% identical to SEQ ID NOs: 42 and 46. In one working example of the present disclosure, the VL region of the recombinant antibody NP-5 scFv or IgG has the amino acid sequence of SEQ ID NO: 42, and the VH region of the recombinant antibody NP-5 scFv or IgG has the amino acid sequence of SEQ ID NO: 46.

Moreover, the CDR-L1, CDR-L2, CDR-L3 of the recombinant antibody NP-6 scFv or IgG respectively comprise the amino acid sequences of SEQ ID NOs: 47, 48 and 49; and the CDR-H1, CDR-H2, and CDR-H3 of the recombinant antibody NP-6 scFv or IgG respectively comprise the amino acid sequences of SEQ ID NOs: 51, 52 and 53. Preferably, the VL region of the recombinant antibody NP-6 scFv or IgG comprises the amino acid sequence at least 80% identical to SEQ ID NO: 50, and the VH region of the recombinant antibody NP-6 scFv or IgG comprises the amino acid sequence at least 80% identical to SEQ ID NO: 54. According to the preferred embodiment, the VL and VH regions of the recombinant antibody NP-6 scFv or IgG respectively comprise the amino acid sequences at least 85% identical to SEQ ID NOs: 50 and 54. More preferably, the VL and VH regions of the recombinant antibody NP-6 scFv or IgG respectively comprise the amino acid sequences at least 90% identical to SEQ ID NOs: 50 and 54. Even more preferably, the VL and VH regions of the recombinant antibody NP-6 scFv or IgG respectively comprise the amino acid sequences at least 95% identical to SEQ ID NOs: 50 and 54. In one working example of the present disclosure, the VL region of the recombinant antibody NP-6 scFv or IgG has the amino acid sequence of SEQ ID NO: 50, and the VH region of the recombinant antibody NP-6 scFv or IgG has the amino acid sequence of SEQ ID NO: 54.

In alternative embodiments, the CDR-L1, CDR-L2, CDR-L3 of the recombinant antibody NP-7 scFv or IgG respectively comprise the amino acid sequences of SEQ ID NOs: 55, 56 and 57; and the CDR-H1, CDR-H2, and CDR-H3 of the recombinant antibody NP-7 scFv or IgG respectively comprise the amino acid sequences of SEQ ID NOs: 59, 60 and 61. Preferably, the VL region of the recombinant antibody NP-7 scFv or IgG comprises the amino acid sequence at least 80% identical to SEQ ID NO: 58, and the VH region of the recombinant antibody NP-7 scFv or IgG comprises the amino acid sequence at least 80% identical to SEQ ID NO: 62. According to the preferred embodiment, the VL and VH regions of the recombinant antibody NP-7 scFv or IgG respectively comprise the amino acid sequences at least 85% identical to SEQ ID NOs: 58 and 62. More preferably, the VL and VH regions of the recombinant antibody NP-7 scFv or IgG respectively comprise the amino acid sequences at least 90% identical to SEQ ID NOs: 58 and 62. Even more preferably, the VL and VH regions of the recombinant antibody NP-7 scFv or IgG respectively comprise the amino acid sequences at least 95% identical to SEQ ID NOs: 58 and 62. In one working example of the present disclosure, the VL region of the recombinant antibody NP-7 scFv or IgG has the amino acid sequence of SEQ ID NO: 58, and the VH region of the recombinant antibody NP-7 scFv or IgG has the amino acid sequence of SEQ ID NO: 62.

Examples of the coronavirus detectable by the present scFv/IgG include, but are not limited to, MERS-CoV, SARS-CoV, and SARS-CoV-2. In one preferred example, the coronavirus is SARS-CoV-2.

According to the embodiments of the present disclosure, the present recombinant antibody is produced by phage-displayed scFv libraries.

According to the embodiments of the present disclosure, the present recombinant antibody in the form of scFv is selected from a phage-displayed scFv library. A phage-displayed scFv library is constructed on a phagemid vector, and the method for construction of a phage-displayed scFv library is well known in the art. For selecting a phage-displayed scFv from the phage-displayed scFv library with high binding affinity and specificity to the nucleocapsid protein of the coronavirus, the method comprises the steps of,

(a) incubating the phage-displayed scFv library having a plurality of phage-displayed scFvs with the nucleocapsid protein of the coronavirus;

(b) purifying the product of step (a) thereby producing a plurality of phage-displayed scFvs, which were respectively bound to the nucleocapsid protein of the coronavirus before the purification; and

(c) repeating at least one run of the steps (a) and (b), each time using the product of the step (b) in previous run as the phage-displayed scFv library for incubating with the nucleocapsid protein of the coronavirus, until the phage-displayed scFv exhibiting the highest binding affinity and specificity to the nucleocapsid protein of the coronavirus is obtained.

In the step (a), the nucleocapsid protein of the coronavirus is preferably immobilized on a solid phase support (e.g., a plate, a bead, a tube, a filter, a chip, and a film, and the like) to facilitate the process of incubation. Non-limiting method used to immobilize the nucleocapsid protein of the coronavirus to the solid phase support includes, covalent immobilization (e.g., amine chemistry, thiol chemistry, carboxyl chemistry, epoxy chemistry, photoactive chemistry, site specific immobilization, diels-alder cycloaddition, click chemistry, and peptide ligation), bioaffinity immobilization (e.g., avidin-biotin system, his-tag system, DNA-directed immobilization, and protein A/protein G-mediated immobilization), and physical immobilization. According to some embodiments, the nucleocapsid protein comprises the amino acid sequence of SEQ ID NO: 1, 2, or 3.

A solid phase support refers to a supporting matter that has a certain volume and shape. Also, the solid phase support is made without particular limitations on the material, volume, or shape, provided it is a solid able to have the nucleocapsid protein of the coronavirus immobilized thereon via the method of immobilization as described above. Specific examples of the solid phase support include: a plate (e.g., plastic plates, glass plates); a bead (e.g., magnetic beads, silica beads, agarose gel beads, polyacrylamide resin beads, latex beads, polystyrene and other plastic beads, ceramic beads, zirconia beads); a tube (e.g., plastic tubes, glass tubes); a filter (e.g., fiberglass filters, filter papers, silica filters); a chip (e.g., plastic chips, glass chips); and a film (e.g., cellulose membranes, nitrocellulose membranes, polyvinylidene fluoride (PVDF) membranes, silica membranes).

In the step (b), the product of the step (a) is purified by carrying out conventional purification procedures known in the art, such as an acid-base neutralization method, in which the product of the step (a) is subject to an acid treatment (e.g., treating with an elution buffer of pH 2.2 (e.g., a HCl/glycine solution)) to separate the scFvs from their respective bound antigens. And the resulting produced phage-displayed scFvs are neutralized by adding an alkaline solution, such as a solution having a pH value of 9.0 (e.g., a Tris-based solution).

For the purpose of selecting an scFv exhibiting highest binding affinity and specificity to the nucleocapsid protein of the coronavirus, the steps (a) and (b) are repeated for at least one run, each time using the alkaline-treated phage-displayed scFvs produced in the previous run as the starting phage library for incubating with the nucleocapsid protein of the coronavirus, until the phage-displayed scFv exhibiting the highest binding affinity and specificity to the nucleocapsid protein of the coronavirus (i.e., the present recombinant antibody) is obtained.

Optionally, the alkaline-treated or neutralized phage-displayed scFvs produced in the step (b) may further be amplified in a host cell, for example, in E. coli, by infecting the host cell with the phage expressing the neutralized scFvs. Then, the amplified phage-displayed scFvs are incubated with the nucleocapsid protein of the coronavirus, and the steps (a) and (b) are repeated, until the phage-displayed scFv exhibiting the highest binding affinity and specificity to the nucleocapsid protein of the coronavirus (i.e., the present recombinant antibody) is obtained.

Once the present recombinant antibody in the form of scFv is obtained from the procedures as described above, it may be engineered to change the format of the antibodies (e.g., Fv, Fab, Fab′, F(ab′)2, diabodies, VHH, IgG) via DNA cloning techniques. DNA encoding the scFv may be easily isolated and sequenced by use of conventional procedures, such as using oligonucleotide probes that are capable of binding specifically to genes encoding the heavy and light chains of the scFv. The phages expressing the scFv serve as a preferred source of such DNA. Once isolated, the DNA may be placed into expression vectors, which are then transfected into host cells such as E. coli cells, simian COS cells or Chinese hamster ovary (CHO) cells, or myeloma cells that do not produce immunoglobulin proteins, to synthesize the desired antibodies in the recombinant host cells.

Depending on desired purposes, the present recombinant antibody may be conjugated with a reporter molecule or a nanoparticle. Exemplary reporter molecules suitable for conjugated with the present recombinant antibody include, but are not limited to, acridine orange, acridine yellow, AP, auramine, benzoxadiazole, bilirubin, biotin, BFP, FAM, cascade blue, cresyl violet, crystal violet, CFP, cyanine, DNA probe, eosin, fluorescein, fluorescein isothiocyanate, GST, GFP, HRP, indocarbocyanine, malachite green, merocyanine, Nile blue, Nile red, nitrobenzoxadiazole, orotidine 5′-phosphate decarboxylase, oxacarbocyanine, peridinin chlorophyll, phycoerythrin, phthalocyanine, porphine, proflavine, pyridyloxazole, RFP, rhodamine, thiacarbocyanine, TRX, and YFP.

Non-limiting examples of nanoparticles include aluminium oxide particle, boron particle, calcium particle, carbon nanotube, cerium oxide particle, clay particle, copper particle, diamond particle, gold particle, graphene particle, hydroxy acid particle, hydroxyapatite particle, iron particle, kojic acid particle, liposome, manganese particle, molybdenum particle, palladium particle, platinum particle, phosphorus particle, potassium particle, silicon dioxide particle, silver particle, sodium silicate particle, titanium dioxide particle, ytterbium trifluoride particle, zinc particle, zinc oxide particle, and zirconium dioxide particle.

2. The Kit

In another aspect of the present disclosure, the present invention provides a kit for detecting a coronavirus; the kit comprises a first and a second recombinant antibodies, in which the first and the second recombinant antibodies are independently selected from the group consisting of the NP-1 to NP-7 scFvs/IgGs. According to one preferred embodiment of the present disclosure, in the present kit, the first recombinant antibody is the NP-3 scFv or IgG; and the second recombinant antibody is any one of the NP-1, NP-2, NP-4, NP-5, NP-6, or NP-7 scFvs/IgGs.

In some further embodiments of the present disclosure, the present kit further comprises a solid phase support, in which one of the first and second recombinant antibodies is linked on the solid phase support directly or indirectly via a linker substance, whereas the other antibody is conjugated with a reporter molecule or a nanoparticle. Choices of the solid phase support, the reporter molecule and the nanoparticle are as described above. Detailed description thereof is omitted herein for the sake of brevity.

In some embodiments, the present kit further comprises an instruction indicating how to use the first and second recombinant antibodies for detecting a coronavirus in accordance with any of the methods as described herein. The instruction supplied in the present kit is typically a written instruction on a label or package insert (e.g., a paper sheet included in the kit), but the machine-readable instruction (e.g., instruction carried on a magnetic or optical storage disk) is also acceptable.

The kits of this invention are provided in suitable packaging. Suitable packaging includes, but is not limited to, vials, bottles, jars, flexible packaging (e.g., sealed Mylar or plastic bags), boxes, and the like. The kits may optionally provide additional components such as buffers and interpretive information. Normally, the kit comprises a container and a label or package insert(s) on or associated with the container. In some embodiments, the invention provides articles of manufacture comprising contents of the kits as described above.

3. The Method for Diagnosing Coronavirus Infection

In another aspect of the present disclosure, the present invention provides a method for determining whether a subject is infected by a coronavirus; the method is performed with a biological sample isolated from the subject, and comprises the step of, detecting the presence or absence of a nucleocapsid protein of the coronavirus in the biological sample by use of the present recombinant antibody or the present kit, wherein the presence of the nucleocapsid protein indicates that the subject is infected by the coronavirus.

The biological sample is preferably obtained from the respiratory tract of the subject; more preferably, the upper respiratory tract of the subject. Non-limiting examples of the biological sample suitable to be used in the present method include, a mucosa tissue (e.g., biopsy, cheek scraping, materials from a nasal swab, or materials from a throat swab) or tissue lysate, a fluid (e.g., bronchoalveolar lavage; nasal, nasopharyngeal, or tracheal wash or aspirate), or a secretion (e.g., saliva, sputum) isolated from the oral cavity, nasal cavity, trachea, bronchus, or lung of the subject. Without bound to the theory, the biological sample may be derived from the other part of the subject, such as blood, plasma, serum, snivel, tears, or urine.

The method for detecting a coronavirus by an antibody (e.g., the present recombinant antibody) is well known in the art, exemplary method includes, but is not limited to, chemiluminescence immunoassay (CLIA), counting immunoassay (CIA), CD/DVD based immunoassay, electrochemiluminescence immunoassay (ECLIA), enzyme-linked immunosorbent assay (ELISA) (including sandwich ELISA), fluoroimmnoassay (FIA), real-time immunoquantitative PCR (iqPCR), magnetic immunoassay (MIA), radioimmunoassay (RIA), and surround optical-fiber immunoassay (SOFIA).

Based on the diagnostic result, a skilled artisan or a clinic practitioner may treat a subject in need thereof (i.e., a subject infected by a coronavirus) by administering an antiviral agent to the subject. So as to alleviate or ameliorate symptoms associated with the infection.

Accordingly, another aspect of the present disclosure pertains to a method of treating a coronavirus infection in a subject. The method comprises,

(a) isolating a biological sample from the subject;

(b) determining the presence or absence of a nucleocapsid protein of the coronavirus in the biological sample of step (a) by use of the present recombinant antibody; and

(c) administering to the subject an effective amount of an antiviral agent based on the result determined by step (b), wherein the nucleocapsid protein of the coronavirus is present in the biological sample of the subject.

Exemplary antiviral agents suitable for treating the coronavirus infection include, but are not limited to, hydroxychloroquine, remdesivir, lopinavir, ritonavir, chloroquine, azithromycin, abacavir, acyclovir, adefovir, amantadine, ampligen, amprenavir, arbidol, atazanavir, atripla, balavir, baloxavir, biktarvy, boceprevir, cidofovir, cobicistat, combivir, daclatasvir, darunavir, delavirdine, descovy, didanosine, docosanol, dolutegravir, doravirine, ecoliever, edoxudine, efavirenz, elvitegravir, emtricitabine, enfuvirtide, entecavir, etravirine, famciclovir, fomivirsen, fosamprenavir, foscamet, fosfonet, ganciclovir, ibacitabine, ibalizumab, idoxuridine, imiquimod, imunovir, indinavir, inosine, interferon, lamivudine, letermovir, loviride, maraviroc, methisazone, moroxydine, nelfinavir, nevirapine, nexavir, nitazoxanide, norvir, oseltamivir, peginterferon, penciclovir, peramivir, pleconaril, podophyllotoxin, pyramidine, raltegravir, ribavirin, rilpivirine, rimantadine, saquinavir, simeprevir, sofosbuvir, stavudine, telaprevir, telbivudine, tenofovir, tipranavir, trifluridine, trizivir, tromantadine, truvada, valaciclovir, valganciclovir, vicriviroc, vidarabine, viramidine, zalcitabine, zanamivir, zidovudine, and a combination thereof.

The antiviral agent may be formulated with different excipients or carriers depending on the intended routes of administration. The present medicament or pharmaceutical composition may be administered intraveneously, intradermally, intraarterially, intraperitoneally, intralesionally, intracranially, intranasally, intrapleurally, intratracheally, intrarectally, topically, intramuscularly, subcutaneoustly, intravesicularlly, intrapericardially, intraocularally, orally, topically, locally, injection, inhalation, infusion, localized perfusion, in any suitable forms such as powders, creams, liquids, aerosols and etc.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

Materials and Methods

1. Cell Cultures

Human embryonic kidney cell line 293 (HEK-293), and Chinese hamster ovary cell line (CHO) were maintained in basic media: Dulbecco's modified Eagle's medium (DMEM) and Kaighn's modification of Ham's F-12 medium (F-12K), respectively, supplemented with 10% fetal bovine serum (FBS), and antibiotics/antimycotics. Cells were grown at 37° C. with a humidified atmosphere of 5% CO2.

2. Preparation of Nucleocapsid Protein

For nucleocapsid protein preparation, DNA sequences encoding the nucleocapsid protein of SARS-CoV-2 (SEQ ID NO: 1), SARS-CoV (SEQ ID NO: 2), and MERS-CoV (SEQ ID NO: 3) were constructed into an E. coli expression vector, respectively, and the constructs were delivered into E. coli competent cells BL21(DE3). The expressed nucleocapsid proteins in the form of the inclusion bodies were harvested, solubilized with 8M urea, and purified with a His-tagged protein purification column. The purity of the purified nucleocapsid proteins was greater than 95% as determined by SDS-PAGE.

3. Phage Display Screening

In the present studies, a scFv phage library was used in the phage display screening procedure. Before biopanning, the scFv phage library was titrated, and the size of the scFv phage library was confirmed as more than 109 clones. The scFv phage library (with 10¹¹-10¹² CFU of PEG-precipitated phages) was screened with the purified nucleocapsid proteins of SARS-CoV-2 (SEQ ID NO: 1), SARS-CoV (SEQ ID NO: 2), and MERS-CoV (SEQ ID NO: 3). Phages that bound to the nucleocapsid proteins were further amplified in E. coli, and were subjected to the next round of biopanning. After two to three rounds of biopanning, a total of 7 phage clones (NP-1 to NP-7) able to bind to the nucleocapsid protein of SARS-CoV-2 were obtained after confirmation by a single colony ELISA assay as follows.

4. Single Colony ELISA

The phage clones NP-1 to NP-7 were propagated in an E. coli host, respectively. Briefly, after each phage clone infected an E. coli host, a single colony of the E. coli with the phage clone was picked up and propagated. For each phage clone, when the E. coli culture reached OD₆₀₀>1, IPTG was added into the cultures at the final concentration of 1 mM to induce secretion of scFv from the phage clone. After incubation at 37° C. overnight, the secreted scFvs (i.e., the NP-1 to NP-7 scFvs) in the supernatant were collected by centrifuge 4,000×g for 10 minutes. The NP-1 to NP-7 scFvs were examined for the binding activity to the nucleocapsid protein by an ELISA assay, and the signal of binding was read by a microplate reader.

5. Transfer of scFv into an IgG Format

For light chain IgG, a mammalian light chain plasmid was constructed by PCR assembling, which contains: an immunoglobulin light chain signal peptide, a light chain variable domain (SEQ ID NOs: 10, 18, 26, 34, 42, 50, or 58), and a light chain constant domain (SEQ ID NO: 63). For heavy chain IgG, a mammalian heavy chain plasmid was also constructed by PCR assembling, which contains: an immunoglobulin heavy chain signal peptide, a heavy chain variable domain (SEQ ID NOs: 14, 22, 30, 38, 46, 54, or 62), and a heavy chain constant domain (SEQ ID NO: 64). Antibodies in the IgG format (i.e., the NP-1 to NP-7 IgGs) were prepared from CHO cells co-transfected with both light and heavy chain plasmids.

6. ELISA Assays

(1) Epitope Competition Analysis

The purified nucleocapsid protein at 0.5 μg/well was coated on an ELISA plate. After blocking, each of the NP-1 to NP-7 IgGs at 1 μg/well was add to each well, respectively. Five minutes later, 100 l of each of the NP-1 to NP-7 scFvs was added to each well, respectively. The scFvs were detected with an anti-c-myc antibody, and the signals were measured with a microplate reader. For each well, the overall signal (including the signal from the IgG and the scFv) became low when the epitopes of the IgG and the scFv were co-localized, whereas the overall signal was not interfered when the epitopes of the IgG and the scFv were not co-localized.

(2) Binding Ability Analysis

For analyzing the binding of the NP-1 to NP-7 IgGs to the mammalian cell-expressed nucleocapsid proteins, plasmids encoding the nucleocapsid protein were transfected into HEK-293 cells, and the cell lysates were prepared from those transfected cells. The NP-1 to NP7 IgGs were diluted to 10, 1, and 0.1 μg/ml in PBS buffer, and coated at 100 l/well on an ELISA plate. After blocking, 100 l of the cell lysates were added into each well of the ELISA plate. The binding was detected with an anti-His antibody, and the signals were measured with a microplate reader.

(3) Measurement of the Concentration of Nucleocapsid Protein

The NP-3 and NP-4 IgGs were respectively diluted to 1 μg/ml in PBS, and coated on an ELISA plate. After blocking with 5% skim milk, both the 293-NP cell lysates and the purified nucleocapsid proteins in serial dilutions (in 5% skim milk; with known concentrations) were added to each well separately. The binding was detected with an anti-His antibody, and the signals were measured with a microplate reader.

### Example Characterization of NP-1 to NP-7 Antibodies

1. Characterization of NP-1 to NP-7 scFvs

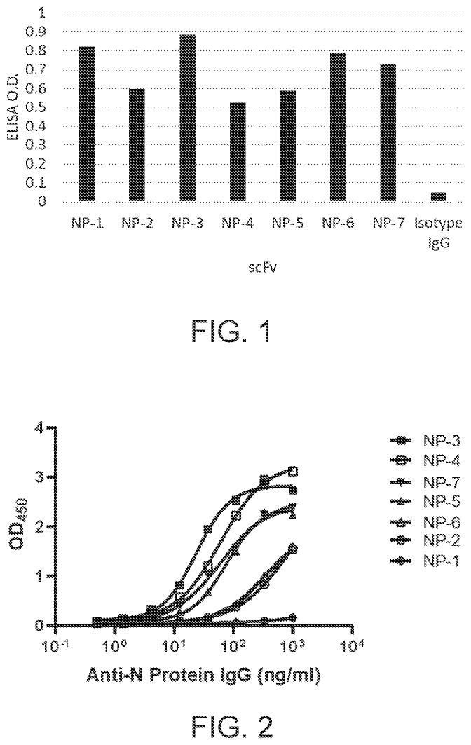

The purpose of this example is to confirm the specificity of the NP-1 to NP-7 scFvs prepared from the phage clones NP-1 to NP-7 to the nucleocapsid protein of the coronavirus expressed by E. coli. As the data depicted in FIG. 1, the NP-1 to NP-7 scFvs exhibited a strong binding affinity toward the nucleocapsid protein of SARS-CoV-2 as compared to the control antibody isotype IgG. Hence, the example demonstrated that the present NP-1 to NP-7 scFvs are useful in detecting SARS-CoV-2.

2. Characterization of NP-1 to NP-7 IgGs

Next, each of the NP-1 to NP-7 scFvs was transferred from the scFv format into the IgG format in accordance with the procedures described in Materials and Methods, in order to enhance the stability of the antibodies and increase flexibility for further applications.

After the antibody format transfer completed, the binding affinity of the thus-obtained NP-1 to NP-7 IgGs to the nucleocapsid protein of SARS-CoV-2 was investigated, in order to make sure if the binding activity of the NP-1 to NP-7 IgGs is preserved.

The binding affinities of NP-1 to NP-7 IgGs toward nucleocapsid protein expressed by E. coli were examined by ELISA. Nucleocapsid proteins were coated on an ELISA plate at the concentration of 0.5 μg/well. After blocking with 5% skim milk, serial diluted IgGs were add to each well. The signals were detected by anti-human antibody. As shown in FIG. 2, NP-1 to NP-7 IgGs exhibited various affinities toward nucleocapsid protein at various concentrations.

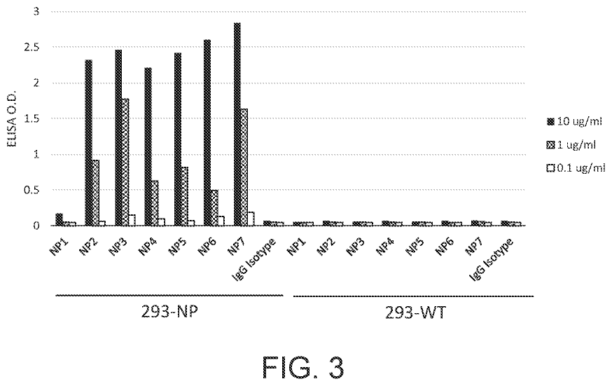

To confirm the binding ability of NP-1 to NP-7 IgGs to nucleocapsid proteins expressed in mammalian cells, the nucleocapsid protein of SARS-CoV-2 was expressed in HEK-293 cells, and the cell lysates from the expressed cells (293-NP), together with the one from the control cells (293-WT), were subjected to an ELISA assay. The results were as depicted in FIG. 3, the NP-1 to NP-7 IgGs at different concentrations (including 10, 1, and 0.1 μg/ml) exhibited a robust binding affinity to 293-NP as compared to 293-WT, indicating that the specificity of the NP-1 to NP-7 IgGs to the nucleocapsid protein of SARS-CoV-2 is preserved, and the NP-1 to NP-7 IgGs provides a potential means to detect SARS-CoV-2.

The epitopes of the nucleocapsid protein for the NP-1 to NP-7 scFvs and IgGs were studied by performing an epitope competition analysis. The results demonstrated that there were at least three different epitopes for the NP-1 to NP-7 scFvs or IgGs (data not shown).

3. Quantification of Nucleocapsid Proteins Using the NP-3 and NP-4 IgGs

According to the analytic results of FIG. 2 and FIG. 3, the NP-3 IgG exhibited a stronger binding affinity, while the NP-4 exhibited a moderate binding affinity, to the nucleocapsid protein of SARS-CoV-2.

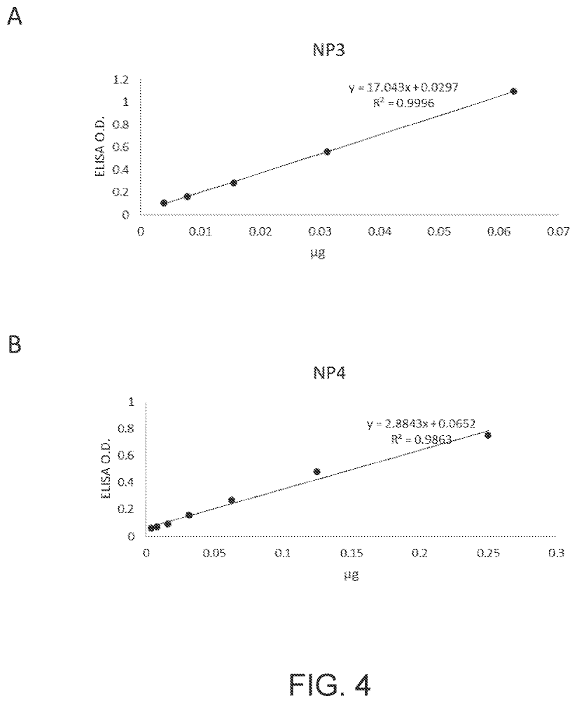

Quantification of the nucleocapsid proteins in the cell lysate 293-NP by the NP-3 and NP-4 IgGs was carried out by an ELISA assay. Standard curves for nucleocapsid protein with known concentration detected by NP-3 and NP-4 IgG were shown in FIG. 4, panels A and B. The results indicated that NP-3 and NP-4 IgG are suitable for antigen detection and quantification.

In sum, the present disclosure has addressed that the present antibodies possess specificity to the coronavirus, and may be a promising biological tool for use in detection of the coronavirus.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant antibody comprising a light chain variable (VL) region and a heavy chain variable (VH) region, wherein the VL region comprises a first complementarity determining region (CDR-L1), a second CDR (CDR-L2), and a third CDR (CDR-L3), and the VH region comprises a first complementarity determining region (CDR-H1), a second CDR (CDR-H2), and a third CDR (CDR-H3), wherein
the CDR-L1, the CDR-L2, the CDR-L3, the CDR-H1, the CDR-H2, and the CDR-H3 respectively comprise the amino acid sequences of SEQ ID NOs: 7, 8, 9, 11, 12, and 13;
the CDR-L1, the CDR-L2, the CDR-L3, the CDR-H1, the CDR-H2, and the CDR-H3 respectively comprise the amino acid sequences of SEQ ID NOs: 15, 16, 17, 19, 20, and 21;
the CDR-L1, the CDR-L2, the CDR-L3, the CDR-H1, the CDR-H2, and the CDR-H3 respectively comprise the amino acid sequences of SEQ ID NOs: 23, 24, 25, 27, 28, and 29;
the CDR-L1, the CDR-L2, the CDR-L3, the CDR-H1, the CDR-H2, and the CDR-H3 respectively comprise the amino acid sequences of SEQ ID NOs: 31, 32, 33, 35, 36, and 37;
the CDR-L1, the CDR-L2, the CDR-L3, the CDR-H1, the CDR-H2, and the CDR-H3 respectively comprise the amino acid sequences of SEQ ID NOs: 39, 40, 41, 43, 44, and 45;
the CDR-L1, the CDR-L2, the CDR-L3, the CDR-H1, the CDR-H2, and the CDR-H3 respectively comprise the amino acid sequences of SEQ ID NOs: 47, 48, 49, 51, 52, and 53; or
the CDR-L1, the CDR-L2, the CDR-L3, the CDR-H1, the CDR-H2, and the CDR-H3 respectively comprise the amino acid sequences of SEQ ID NOs: 55, 56, 57, 59, 60, and 61.

2. The recombinant antibody of claim 1, wherein the CDR-L1, the CDR-L2, the CDR-L3, the CDR-H1, the CDR-H2, and the CDR-H3 respectively comprise the amino acid sequences of SEQ ID NOs: 23, 24, 25, 27, 28, and 29.

3. The recombinant antibody of claim 1, wherein
the VL and the VH regions respectively comprise the amino acid sequences of SEQ ID NOs: 10 and 14;
the VL and the VH regions respectively comprise the amino acid sequences of SEQ ID NOs: 18 and 22;
the VL and the VH regions respectively comprise the amino acid sequences of SEQ ID NOs: 26 and 30;
the VL and the VH regions respectively comprise the amino acid sequences of SEQ ID NOs: 34 and 38;
the VL and the VH regions respectively comprise the amino acid sequences of SEQ ID NOs: 42 and 46;
the VL and the VH regions respectively comprise the amino acid sequences of SEQ ID NOs: 50 and 54; or
the VL and the VH regions respectively comprise the amino acid sequences of SEQ ID NOs: 58 and 62.

4. The recombinant antibody of claim 3, wherein the VL and the VH regions respectively comprise the amino acid sequences of SEQ ID NOs: 26 and 30.

5. The recombinant antibody of claim 1, wherein the recombinant antibody is conjugated with a reporter molecule or a nanoparticle.

6. The recombinant antibody of claim 5, wherein the reporter molecule is acridine orange, acridine yellow, alkaline phosphatase (AP), auramine, benzoxadiazole, bilirubin, biotin, blue fluorescent protein (BFP), 6′-carboxyfluorescein (FAM), cascade blue, cresyl violet, crystal violet, cyan fluorescent protein (CFP), cyanine, DNA probe, eosin, fluorescein, fluorescein isothiocyanate, glutathione-S-transferase (GST), green fluorescence protein (GFP), horseradish peroxidase (HRP), indocarbocyanine, malachite green, merocyanine, Nile blue, Nile red, nitrobenzoxadiazole, orotidine 5′-phosphate decarboxylase, oxacarbocyanine, peridinin chlorophyll, phycoerythrin, phthalocyanine, porphine, proflavine, pyridyloxazole, red fluorescent protein (RFP), rhodamine, thiacarbocyanine, thioredoxin (TRX), or yellow fluorescent protein (YFP).

7. The recombinant antibody of claim 5, wherein the nanoparticle is aluminium oxide particle, boron particle, calcium particle, carbon nanotube, cerium oxide particle, clay particle, copper particle, diamond particle, gold particle, graphene particle, hydroxy acid particle, hydroxyapatite particle, iron particle, kojic acid particle, liposome, manganese particle, molybdenum particle, palladium particle, platinum particle, phosphorus particle, potassium particle, silicon dioxide particle, silver particle, sodium silicate particle, titanium dioxide particle, ytterbium trifluoride particle, zinc particle, zinc oxide particle, or zirconium dioxide particle.

8. The recombinant antibody of claim 1, wherein the recombinant antibody is specific to severe acute respiratory syndrome coronavirus 2 (SARS-CoV-2).

9. A kit for a coronavirus detection comprising a first recombinant antibody and a second recombinant antibody, independently selected from the recombinant antibody of claim 1.

10. The kit of claim 9, wherein the coronavirus is SARS-CoV-2.

11. The kit of claim 9, wherein the CDR-L1, the CDR-L2, the CDR-L3, the CDR-H1, the CDR-H2, and the CDR-H3 of the first recombinant antibody respectively comprise the amino acid sequences of SEQ ID NOs: 23, 24, 25, 27, 28, and 29; and
the CDR-L1, the CDR-L2, the CDR-L3, the CDR-H1, the CDR-H2, and the CDR-H3 of the second recombinant antibody respectively comprise the amino acid sequences of SEQ ID NOs: 7, 8, 9, 11, 12, and 13;
the CDR-L1, the CDR-L2, the CDR-L3, the CDR-H1, the CDR-H2, and the CDR-H3 of the second recombinant antibody respectively comprise the amino acid sequences of SEQ ID NOs: 15, 16, 17, 19, 20, and 21;
the CDR-L1, the CDR-L2, the CDR-L3, the CDR-H1, the CDR-H2, and the CDR-H3 of the second recombinant antibody respectively comprise the amino acid sequences of SEQ ID NOs: 31, 32, 33, 35, 36, and 37;
the CDR-L1, the CDR-L2, the CDR-L3, the CDR-H1, the CDR-H2, and the CDR-H3 of the second recombinant antibody respectively comprise the amino acid sequences of SEQ ID NOs: 39, 40, 41, 43, 44, and 45;
the CDR-L1, the CDR-L2, the CDR-L3, the CDR-H1, the CDR-H2, and the CDR-H3 of the second recombinant antibody respectively comprise the amino acid sequences of SEQ ID NOs: 47, 48, 49, 51, 52, and 53; or
the CDR-L1, the CDR-L2, the CDR-L3, the CDR-H1, the CDR-H2, and the CDR-H3 of the second recombinant antibody respectively comprise the amino acid sequences of SEQ ID NOs: 55, 56, 57, 59, 60, and 61.

12. The kit of claim 11, wherein the VL and the VH regions of the first recombinant antibody respectively comprise the amino acid sequences of SEQ ID NO: 26 and 30; and
the VL and the VH regions of the second recombinant antibody respectively comprise the amino acid sequences of SEQ ID NO: 10 and 14;
the VL and the VH regions of the second recombinant antibody respectively comprise the amino acid sequences of SEQ ID NO: 18 and 22;
the VL and the VH regions of the second recombinant antibody respectively comprise the amino acid sequences of SEQ ID NO: 34 and 38;
the VL and the VH regions of the second recombinant antibody respectively comprise the amino acid sequences of SEQ ID NO: 42 and 46;
the VL and the VH regions of the second recombinant antibody respectively comprise the amino acid sequences of SEQ ID NO: 50 and 54; or
the VL and the VH regions of the second recombinant antibody respectively comprise the amino acid sequences of SEQ ID NO: 58 and 62.

13. The kit of claim 9, further comprising a solid phase support, wherein one of the first and the second recombinant antibodies is linked to the solid phase support, and the other is conjugated with a reporter molecule or a nanoparticle.

14. The kit of claim 13, wherein the solid phase support is selected from the group consisting of a plate, a bead, a tube, a filter, a chip, and a film.

15. A method of determining whether a subject is infected by a coronavirus via a biological sample isolated from the subject, comprising detecting the presence or absence of a nucleocapsid protein of the coronavirus in the biological sample by use of the recombinant antibody of claim 1, wherein the presence of the nucleocapsid protein indicates that the subject is infected by the coronavirus.

16. The method of claim 15, wherein the coronavirus is SARS-CoV-2.

17. The method of claim 15, wherein the subject is a human.
